(19) United States
(12) Patent Application Publication
(10) Pub. No.: US 2022/0167915 A1
(43) Pub. Date: Jun. 2, 2022

(54) STRESS ESTIMATION APPARATUS, STRESS ESTIMATION METHOD, AND COMPUTER READABLE RECORDING MEDIUM

(71) Applicant: NEC Corporation, Minato-ku, Tokyo (JP)

(72) Inventors: Yoshiki NAKASHIMA, Tokyo (JP); Yoshifumi ONISHI, Tokyo (JP)

(73) Assignee: NEC Corporation, Minato-ku, Tokyo (JP)

(21) Appl. No.: 17/600,199

(22) PCT Filed: Mar. 30, 2020
(86) PCT No.: PCT/JP2020/014444

§ 371 Date: Sep. 30, 2021

(30) Foreign Application Priority Data
Apr. 8, 2019 (JP) 2019-073714

(51) Int. Cl. A61B 5/00; A61B 5/16

(52) U.S. Cl. CPC A61B 5/4884; A61B 5/165

(57) ABSTRACT

A stress estimation apparatus 1 acquires a biosignal of a measurement subject, which includes acceleration information of an action of at least one part of the measurement subject. A moving average is calculated from the acceleration information included in the acquired biosignal, and a threshold of an activity state of the measurement subject is calculated from the calculated moving average. Then, an activity state in a specific period is identified from the calculated moving average and the calculated threshold. Per-activity state data is generated by categorizing chronological data pieces of the acquired biosignal other than the acceleration information by identified activity state, and stress is estimated from the generated per-activity state data.

### TECHNICAL FIELD

The present invention relates to a stress estimation apparatus and a stress estimation method that use a biosignal, and further relates to a computer readable recording medium that includes a program recorded thereon, the program being intended to realize these apparatus and method.

### BACKGROUND ART

Recent years have witnessed a problem in which autonomic nerves are impaired by a long duration of a state where sympathetic nerves have become active due to, for example, exposure to a long-term stressor (physical/psychological factor that causes stress), thereby harming mental health. Therefore, a technique has been proposed in which stress of a measurement subject is monitored by making the measurement subject wear a wearable terminal on a daily basis and constantly measuring biosignals, which are signals reflecting biological information (the amount of perspiration, the temperature of skin surface, body movements, and the like) of the measurement subject, from the wearable terminal. With such a technique, in order to identify whether the reaction of the biological information reflected on the biosignals being measured is caused by a physical activity, such as intense exercise, or is attributed to a mental activity, such as stress, it is generally necessary to estimate the activity states of the body of the measurement subject with use of, for example, a signal of an acceleration meter. Examples of the activity states of the body include a seated state (seated position), a walking state (walking), a running state (running), and so forth.

Non-Patent Document 1 discloses a technique to identify the three activity states including the seated position, walking, and running from 30 days' worth of data of 20 people based on Activity Magnitude (hereinafter abbreviated as AM). AM is the moving average of changes in the RMS (Rooted Mean Square) of triaxial acceleration. Next, the average, variance, median value, and constituent elements of a histogram of the power spectral density of perspiration and body movements, for each of the three estimated activity states including the seated position, running, and walking, are calculated as feature values, and stress is estimated with use of machine learning while using stress values that have been quantified by a survey about stress as correct answer labels. However, thresholds of AM for estimating the states of the seated position, walking, and running are defined as common numerical values that do not change with each individual.

On the other hand, Non-Patent Document 2 discloses a technique for counting, on a pedometer, each step taken while walking. Regarding this technique, for example, a technique is disclosed that identifies each step taken while walking with use of a threshold that has been calculated as an average of the maximum value and the minimum value of the RMSs of acceleration during the immediately preceding second. Although this technique cannot distinguish between walking and running, it can be used as a technique to distinguish between the seated position and walking, or between the seated position and running.

### LIST OF RELATED ART DOCUMENTS

### Non-Patent Document

Non-Patent Document 1: A. Sano, “Measuring College Students' Sleep, Stress, Mental Health and Wellbeing with Wearable Sensors and Mobile Phones”, Massachusetts Institute of Technology, 2015.
Non-Patent Document 2: VINCENZO GENOVESE, ANDREA MANNINI, AND ANGELO M. SABATINI,” A Smartwatch Step Counter for Slow and Intermittent Ambulation”, SPECIAL SECTION ON BODY AREA NETWORKS, IEEE, 2017

### SUMMARY OF INVENTION

### Problems to be Solved by the Invention

Meanwhile, in Non-Patent Document 1, certain fixed thresholds are defined that have been calculated in advance using data of AM with activity state labels added thereto, and individual differences cannot be reflected in connection with identification of an activity state. On the other hand, although the technique disclosed in Non-Patent Document 2 allows individual differences to be reflected because the threshold is changed constantly, Non-Patent Document 2 pertains only to a technique to distinguish between the seated position and walking or distinguish between the seated position and running, and does not provide a technique to distinguish between walking and running.

An example object of the present invention is to provide a technique with which stress of a measurement subject can be determined by identifying the activity states of the measurement subject.

### Means for Solving the Problems

In order to achieve the aforementioned object, a stress estimation apparatus according to an example aspect of the present invention is a stress estimation apparatus that estimates stress of a measurement subject, the stress estimation apparatus characterized by including: a signal acquisition unit configured to acquire a biosignal, including acceleration information of an action of at least one part of the measurement subject; a moving average calculation unit configured to calculate a moving average from the acceleration information included in the biosignal acquired by the signal acquisition unit; a threshold calculation unit configured to calculate a threshold of an activity state of the measurement subject from the moving average calculated by the moving average calculation unit; an activity state identification unit configured to identify an activity state in a specific period from the moving average calculated by the moving average calculation unit and the threshold calculated by the threshold calculation unit; a per-activity state data generation unit configured to generate per-activity state data by categorizing chronological data pieces of the biosignal other than the acceleration information by activity state identified by the activity state identification unit, the biosignal having been acquired by the signal acquisition unit; and a stress estimation unit configured to estimate stress from the per-activity state data generated by the per-activity state data generation unit.

Also, in order to achieve the aforementioned object, a stress estimation method according to an example aspect of the present invention is a stress estimation method that estimates stress of a measurement subject, the stress estimation method characterized by including: a step of acquiring a biosignal, including acceleration information of an action of at least one part of the measurement subject; a step of calculating a moving average from the acceleration information included in the acquired biosignal; a step of calculating a threshold of an activity state of the measurement subject from the calculated moving average; a step of identifying an activity state in a specific period from the calculated moving average and the calculated threshold; a step of generating per-activity state data by categorizing chronological data pieces of the acquired biosignal other than the acceleration information by identified activity state; and a step of estimating stress from the generated per-activity state data.

Furthermore, in order to achieve the aforementioned object, a program according to an example aspect of the present invention is a computer readable recording medium characterized by including a program recorded thereon, the program causing a computer to estimate stress of a measurement subject and including instructions for causing the computer to carry out: a step of acquiring a biosignal, including acceleration information of an action of at least one part of the measurement subject; a step of calculating a moving average from the acceleration information included in the acquired biosignal; a step of calculating a threshold of an activity state of the measurement subject from the calculated moving average; a step of identifying an activity state in a specific period from the calculated moving average and the calculated threshold; a step of generating per-activity state data by categorizing chronological data pieces of the acquired biosignal other than the acceleration information by identified activity state; and a step of estimating stress from the generated per-activity state data.

### Advantageous Effects of the Invention

According to the present invention, stress of a measurement subject can be determined by identifying the activity states of the measurement subject.

### BRIEF DESCRIPTION OF THE DRAWINGS

FIG. 1 is a block diagram showing a schematic configuration of a stress estimation apparatus of an example embodiment.

FIG. 2 is a block diagram showing a configuration of the stress estimation apparatus in a specific manner.

FIG. 3 is a diagram showing a virtual histogram of a moving average.

FIG. 4 is a diagram showing a histogram of moving averages calculated from one month's worth of data of four measurement subjects.

FIG. 5 is a diagram showing an hourly moving average in one day.

FIG. 6 is a diagram of a numerical value histogram of the moving average in one day shown in FIG. 5.

FIG. 7 is a histogram of one month's worth of moving average of a measurement subject shown in FIG. 5 and FIG. 6.

FIG. 8 is a diagram obtained by extracting only a portion that includes two regions with concave down from a histogram that includes a plurality of regions with concave down as shown in FIG. 7.

FIG. 9 is a diagram showing the result of applying logarithmic conversion processing to the histogram of FIG. 7.

FIG. 10 is a flow diagram showing the operations of the stress estimation apparatus.

FIG. 11 is a block diagram showing one example of a computer that realizes the stress estimation apparatus in the example embodiment.

### EXAMPLE EMBODIMENT

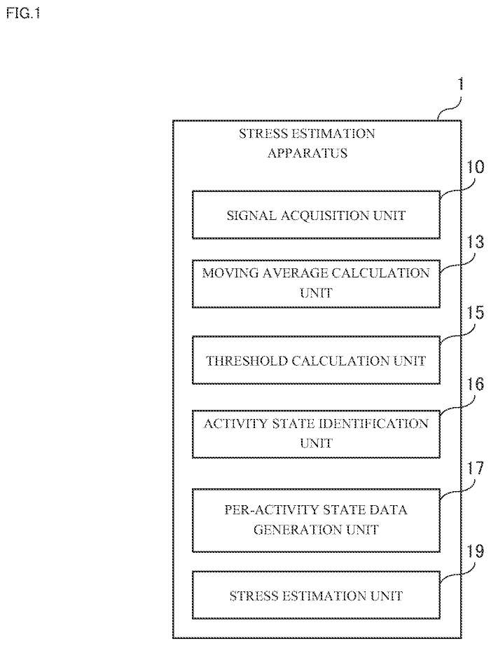

The following describes a configuration of a stress estimation apparatus in one example embodiment of the present invention with reference to FIG. 1 to FIG. 11.

[Description of Configuration]

FIG. 1 is a block diagram showing a schematic configuration of a stress estimation apparatus 1 in the present example embodiment.

The stress estimation apparatus 1 is an apparatus that estimates stress of a measurement subject. The stress estimation apparatus 1 includes a signal acquisition unit 10, a moving average calculation unit 13, a threshold calculation unit 15, an activity state identification unit 16, a per-activity state data generation unit 17, and a stress estimation unit 19.

The signal acquisition unit 10 acquires biosignals of a measurement subject, which include acceleration information of an action of at least one part of the measurement subject.

The moving average calculation unit 13 calculates a moving average from the acceleration information included in the biosignals acquired by the signal acquisition unit 10.

The threshold calculation unit 15 calculates a threshold of an activity state of the measurement subject from the moving average calculated by the moving average calculation unit 13.

The activity state identification unit 16 identifies an activity state in a specific period from the moving average calculated by the moving average calculation unit 13 and the threshold calculated by the threshold calculation unit 15.

The per-activity state data generation unit 17 generates data of each activity state by categorizing chronological data pieces of the biosignals other than the acceleration information, which have been acquired by the signal acquisition unit 10, by activity state identified by the activity state identification unit 16.

The stress estimation unit 19 estimates stress from the biosignal signals categorized by the per-activity state data generation unit 17.

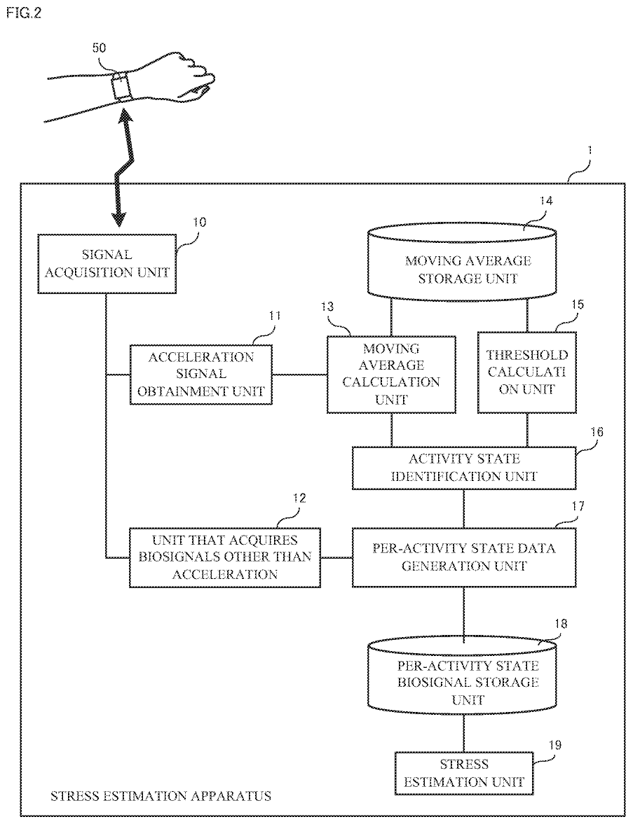

Next, the configuration of the stress estimation apparatus 1 in the present example embodiment will be described more specifically with reference to FIG. 2. FIG. 2 is a block diagram showing the configuration of the stress estimation apparatus 1 in a specific manner.

The stress estimation apparatus 1 is composed of a computer. In the present example embodiment, the stress estimation apparatus 1 is capable of performing wired or wireless data communication with a wearable terminal 50 mounted on a part of the body of the measurement subject, such as an arm. Note that the wearable terminal 50 may perform data communication with a mobile device terminal (e.g., smartphone) owned by the measurement subject via, for example, wireless near-field communication, and the stress estimation apparatus 1 and the wearable terminal 50 may perform data communication with each other via this mobile device terminal.

The wearable terminal 50 measures biological information of the measurement subject, and outputs biosignals that reflect such biological information. Examples of the biological information include the amount of perspiration, the temperature of skin surface, body movements, the pulse rate, the heart rate, and the respiratory rate of the measurement subject. Note that the biological information is not limited to these, and it is sufficient for the biological information to be information with which a mental state (e.g., stress) of the measurement subject can be estimated, such as information that reflects the activity of autonomic nerves of the measurement subject.

Also, the wearable terminal 50 includes an acceleration sensor. The acceleration sensor outputs detected acceleration information as an acceleration signal. The acceleration signal is included in the biosignals because the acceleration information reflects the body movements of the measurement subject and the body movements per se are included in the biological information.

The wearable terminal 50 obtains signals of skin conductivity, triaxial acceleration, and the temperature of skin surface, respectively, at a certain sampling rate, and stores them to a built-in memory. Note that it is sufficient for the wearable terminal 50 to be wearable on the measurement subject as a badge type, an employee ID card type, an earphone type, and a shirt type other than a wristband type, and to be capable of measuring one of the biosignals reflecting the biological information and the acceleration signal.

The wearable terminal 50 may output biosignals each time biological information is measured, or may output biosignals after accumulating biological information that has been measured in a certain period.

The stress estimation apparatus 1 includes the signal acquisition unit 10, an acceleration signal obtainment unit 11, a unit that acquires biosignals other than acceleration 12, the moving average calculation unit 13, a moving average storage unit 14, the threshold calculation unit 15, the activity state identification unit 16, the per-activity state data generation unit 17, a per-activity state biosignal storage unit 18, and the stress estimation unit 19.

The signal acquisition unit 10 acquires biosignals including an acceleration signal, which are output from the wearable terminal 50, at any time.

The acceleration signal obtainment unit 11 obtains the acceleration signal included in the biosignals acquired by the signal acquisition unit 10. The unit that acquires biosignals other than acceleration 12 acquires, among the biosignals acquired by the signal acquisition unit 10, biosignals other than the acceleration signal.

The moving average calculation unit 13 calculates a moving average related to chronological changes in the acceleration signal obtained by the acceleration signal acquisition unit 11. Provided that the moving average calculated by the moving average calculation unit 13 is denoted by RMS_{ACC}, the moving average RMS_{ACC} is calculated from, for example, the following expression (1).

$$\begin{matrix}[ Math . 1 ] & \\ RM {S}_{A C C} ( {t}_{2} ) = \frac{1}{{T}_{2}} {\sum }_{{t}_{1} = {t}_{2} - {T}_{2}}^{{t}_{2}} \sqrt{{( {\overline{a}}_{{x}_{1} {t}_{1}} )}^{2} + {( {\overline{a}}_{{x}_{2} {t}_{1}} )}^{2} + {( {\overline{a}}_{{x}_{3} {t}_{1}} )}^{2}} & ( 1 ) \\ [ Math . 2 ] & \\ {\overline{a}}_{{x}_{i} {t}_{1}} = {a}_{{x}_{i} {t}_{1}} - \frac{1}{{T}_{1}} {\sum }_{{t}_{0} = {t}_{1} - {T}_{1}}^{{t}_{1}} {a}_{{x}_{i} {t}_{{0}^{′}}} ( i = 1 , 2 , 3 ) & ( 2 )\end{matrix}$$

Here, a denotes acceleration. x₁, x₂, and x₃ denote three axes (x axis, y axis, and z axis) in the space. a with xᵢ (i=1, 2, 3) added thereto as subscript indexes denotes an acceleration component in each of the directions of the three axes. Also, t₀, t₁, and t₂ (t₀<t₁<t₂) denote times at which the wearable terminal 50 detected acceleration. t₁ is represented by t₀+T₁, and t₂ is represented by t₁+T₂.

The aforementioned expression (2) indicates a degree of change in acceleration at time t₁, which is obtained by comparing acceleration at time t₁ with the moving average of acceleration from time t₀ to time t₁. Also, expression (1) calculates the moving average, at time t₁, of the root mean square (RMS) of the degree of change in acceleration from time t₁ to time t₂.

By calculating the moving average RMS_{ACC} from the aforementioned expression (1), the average value of changes in acceleration in a certain time frame (from time t₁ to time t₂) can be calculated, rather than an instantaneous change in acceleration. In this way, later-described activity states can be determined unerringly. Note that the foregoing is one example of a method of calculating the moving average, and the numerical values that are calculated for a similar purpose are all included as the foregoing “moving average”.

Each time the moving average calculation unit 13 calculates a moving average, the moving average storage unit 14 stores this moving average.

Based on the moving average corresponding to a certain period (e.g., 4 weeks) stored in the moving average storage unit 14, the threshold calculation unit 15 calculates a threshold of the moving average of an activity state of each individual measurement subject. The threshold of the moving average is a value used by the later-described activity state identification unit 16 in identifying an activity state. The following describes the threshold calculated by the threshold calculation unit 15 and calculation processing thereof.

First, the relationship between a moving average and an activity state will be described.

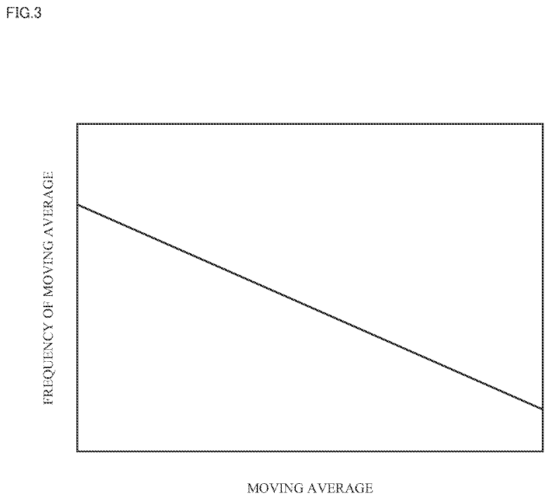

The numerical value of the moving average calculated by the moving average calculation unit 13 increases in a case where the activity of the measurement subject is intense, such as when the measurement subject is running. On the other hand, this numerical value decreases in a case where the activity of the measurement subject is not intense. The frequency of the case where the activity becomes intense is generally lower than the frequency of the case where the activity is not intense. Therefore, from a drawing of a histogram of the moving average in a certain period (e.g., one day), it can be predicted that the frequency decreases as the numerical value of the moving average increases as shown in FIG. 3. FIG. 3 is a diagram showing a virtual histogram of a moving average.

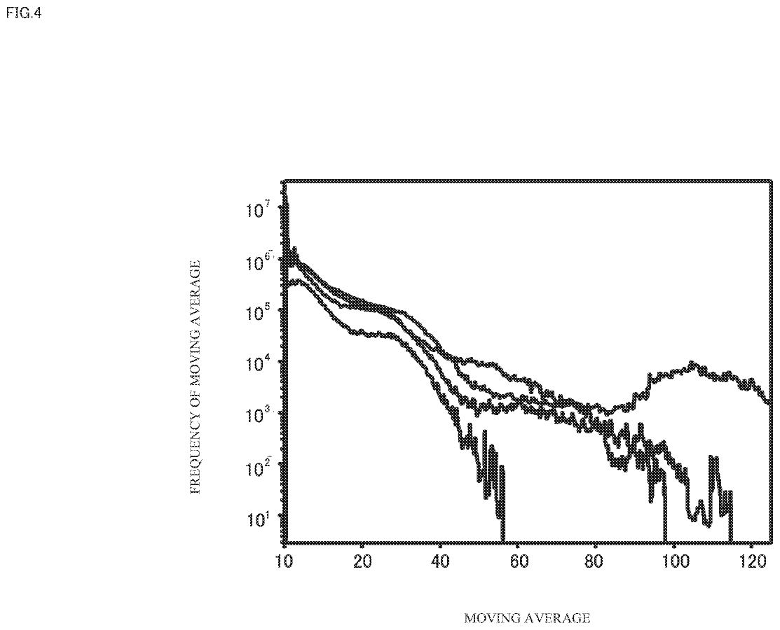

However, when the moving averages of a plurality of measurement subjects are calculated, it is understood that a decrease in a moving average is not constant as shown in FIG. 4. FIG. 4 is a diagram showing a histogram of the moving averages calculated from one month's worth of data of four measurement subjects. As shown in FIG. 4, the moving averages decrease via several inflection points (points at which the sign (positive and negative) of the curvature of the graph is reversed). Furthermore, in the case of any test subject, a region delimited by inflection points is a region with concave down (a portion with a negative curvature, or a portion in which the tangent line is above a curved line), or a region with concave up (a portion with a positive curvature, or a portion in which the tangent line is below a curved line). Among these, a region with concave down is a portion in which the frequency of the numerical value of the moving average is relatively high. In other words, this is a portion in which the frequency of intensity of a specific activity is relatively high.

The present inventors have considered that below is the reason why inflection points appear as shown in FIG. 4 in a drawing of a graph of a histogram of a moving average, without the occurrence of a constant decrease shown in FIG. 3. In human activities, such as daytime activities of an office worker, it is thought that a plurality of activity states exist; for example, the office worker is mostly in a seated position (a seated state), occasionally walking (in a walking state), and running (in a running state) on rare occasion. It can be estimated that, in each activity state, the numerical value of the moving average thereof is concentrated in a certain region. This is because the intensity of an activity when an individual is in a seated position, walking, or running (the intensity of the way an individual swings his/her arm when he/she is wearing the wearable terminal on the arm) tends to have a roughly constant value unless he/she consciously changes the intensity (e.g., unless he/she intentionally swings the arm quickly or slowly while walking). It can therefore be estimated that, in a drawing of a histogram of a moving average in a certain period, a region with concave down is formed in correspondence with each activity state. For the aforementioned reason, it is thought that inflection points appear as shown in FIG. 4 in a drawing of a graph of a histogram of a moving average.

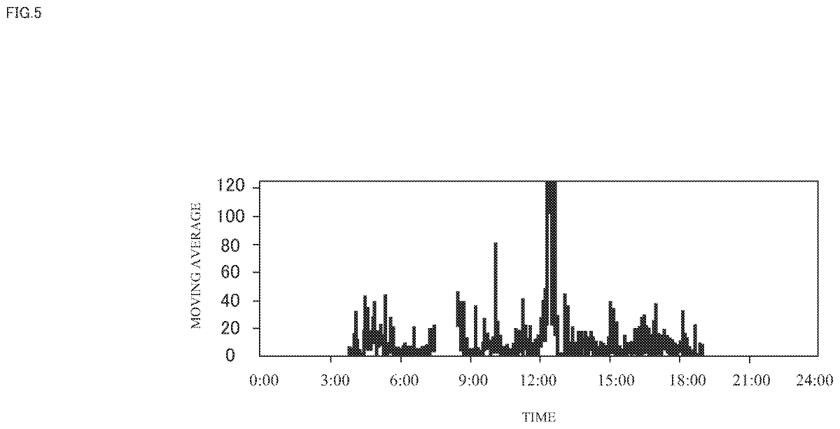

FIG. 5 is obtained by actually applying the moving average calculated by the moving average calculation unit 13 to one day's worth of acceleration data of a certain measurement subject with activity state labels added thereto (with known activity states), and plotting the result of the application as chronological data pieces. FIG. 5 is a diagram showing a moving average per time in one day. In this example, it is assumed that the measurement subject is jogging (the activity state is running) between 12:10 and 12:30, and having a meeting (the activity state is a seated position) between 13:00 and 15:00. It is also assumed that, on this day, the measurement subject has not stood in a stationary fashion for a certain period, and has only walked to move in the office as an activity state that is neither a seated position nor running. In this case, the numerical value of the moving average in a time frame between 12:10 and 12:30 is concentrated around 90 to 120, and the numerical value of the moving average in a time frame between 13:00 and 15:00 is concentrated around 0.

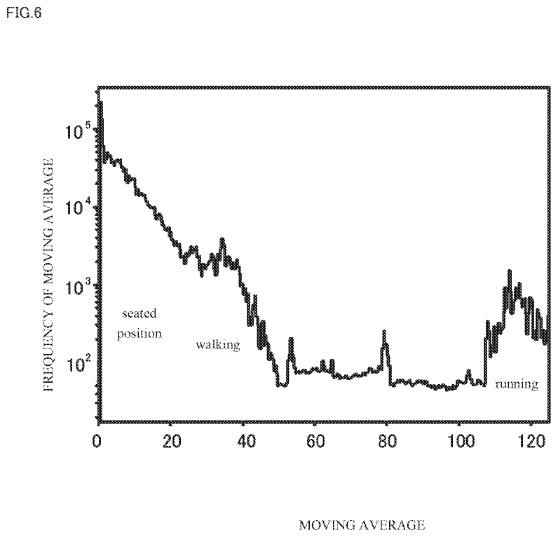

FIG. 6 is a diagram of a numerical value histogram of the moving average in one day shown in FIG. 5.

As shown in FIG. 6, there is one region with concave down around the moving average with a numerical value of 0, there is also one region with concave down around 90 to 120, and there is also one region with concave down therebetween. It can be estimated from FIG. 5 that the activity state in the region in which the numerical value of the moving average is around 0 is the “seated position”, the activity state in the region around 90 to 120 is “running”, and the activity state in the region therebetween is “running” By thus accumulating data pieces of the moving average and forming a histogram thereof, a region with concave down is formed in correspondence with each activity state as stated earlier, and beneficial information for estimating the activity states of individuals on an individual basis is obtained.

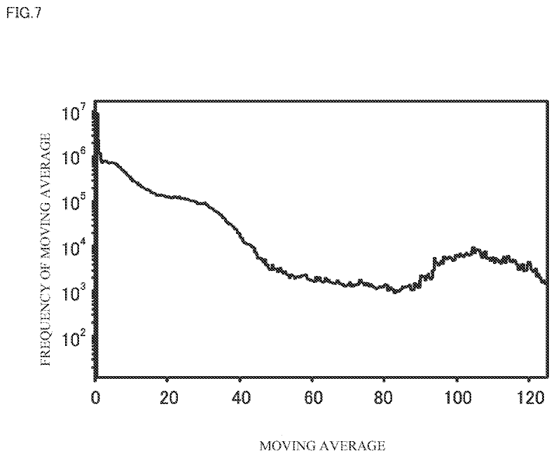

FIG. 7 is a histogram of one month's worth of moving average related to changes in acceleration of the measurement subject shown in FIG. 5 and FIG. 6. In this histogram, too, one region with concave down can be confirmed in each of the region around 0, the region around 90 to 120, and the region therebetween. Based on the estimation that has been made with respect to one day's worth of data of FIG. 6, it can be estimated, also for FIG. 7, that the activity state in the region in which the numerical value of the moving average is around 0 is the “seated position”, the activity state in the region around 90 to 120 is “running”, and the activity state in the region therebetween is “running”.

In this way, in a case where the main activity states include three states, namely the seated position, walking, and running, three regions with concave down that respectively correspond to the activity states are formed also in a histogram of the moving average. In the present example embodiment, the three activity states including the seated position, walking, and running are used similarly to FIG. 5, assuming daytime activities of an office worker.

However, the number of the activity states is not limited to three. Also, the types of the activity states are not limited to the seated position, walking, and running. For example, it is sufficient for the activity states to be states that can be considered as daily activities of a human, such as a standing state (i.e., an upright position) and a lying state (e.g., a recumbent position). Furthermore, with regard to an occupation that involves driving of a vehicle, there is a possibility that the activity state is the seated position but the numerical value of the moving average related to changes in acceleration differs from the case of the seated position on a stationary chair, and this activity state (driving) can be defined as another activity state. As described above, various activity states (an upright position, a recumbent position, driving, and so forth) can be envisioned.

Next, thresholds will be described.

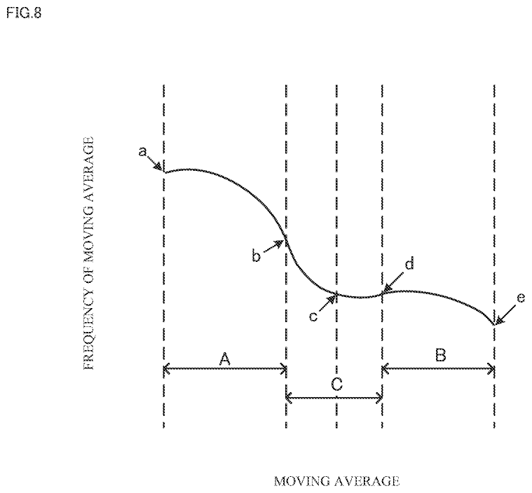

FIG. 8 is a diagram obtained by extracting only a portion that includes two regions with concave down from a histogram that includes a plurality of regions with concave down as shown in FIG. 7. Assume a case where the activity states in two regions A and B with concave down are to be identified from this FIG. 8.

In a case where only regions with concave down are to be identified, the later-described activity state identification unit 16 can regard the region A as a first region with concave down by obtaining an inflection point a and an inflection point b, regard the region B as a second region with concave down by obtaining an inflection point d and an inflection point e, and identify the activity states by applying a specific activity state to each region with concave down. In this case, a region C with concave up remains as a region with an undetermined activity state. In the present example embodiment, data of the region C is also used as data of one of the activity states for the purpose of estimating stress by making use of as much data as possible. To this end, the inflection point b, the inflection point d, a midpoint c therebetween, or the like can be set as a boundary. Among these, the inflection point b or the inflection point d, which can be calculated directly as an extremum of a first derivative, is mathematically easy to obtain. While the inflection point b and the inflection point d can both be set as boundaries for identifying an activity state, the inflection point d is used as a boundary point in the present example embodiment. In a graph of a first derivative, the inflection point d is a maximum (the inflection point b is a minimum).

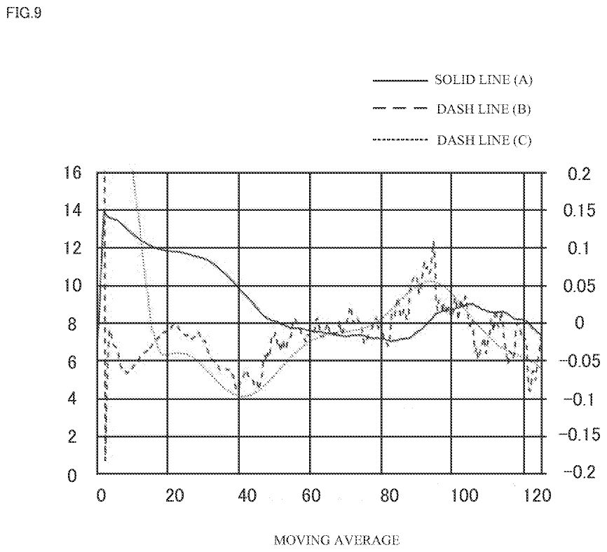

FIG. 9 is a diagram showing the result of applying logarithmic conversion processing to the histogram of FIG. 7. In FIG. 9, the result of applying the logarithmic conversion processing to the histogram of FIG. 7 is indicated by a solid line (A).

Performing the logarithmic conversion processing as indicated by the solid line (A) alleviates the difference between the frequency of a case where the activity state is not intense and the frequency of a case where the activity state is intense, which brings about the result that is appropriate for analysis. However, even in a case where the logarithmic conversion processing is not performed, or a case where a monotonically increasing function other than a logarithm is applied, implementation is possible in a similar procedure. Note that the monotonically increasing function indicates a function which, with regard to a function F(x), satisfies the relationship F(x1)>F(x2) when x1>x2 and the relationship F(x1)<F(x2) when x1<x2, and which does not change a magnitude relationship even if conversion is performed by applying this function. y=log(x) satisfies these conditions. This solid line (A) is equivalent to the curve of FIG. 8. As stated earlier in connection with the description of FIG. 8, in the present example embodiment, a point equivalent to the inflection point d of FIG. 8 is obtained by obtaining a maximum through derivative calculation on a first derivative of the solid line (A), and the position of this point is used as a threshold of an activity state.

In order to obtain a maximum of a graph of a linear function of the solid line (A), a graph of a first derivative of the solid line (A) is drawn as indicated by a dash line (B). However, if the dash line (B) is left as is, smoothing is not sufficient, and a large number of maxima exist. Therefore, the dash line (B) is smoothed as indicated by a dash line (C). For example, a moving average is obtained using a pre-designated window width. On the dash line (C), a maximum appears at a point at which the numerical value of the moving average has slightly exceeded 20, and a point around 90. These points of maxima are used as a threshold between a seated position and walking of a measurement subject and a threshold between walking and running of the measurement subject, in order from a position close to a zero point of the moving average.

In the foregoing manner, the threshold calculation unit 15 calculates thresholds from the acceleration signal.

Note that in the histogram of the moving average, a boundary can be obtained using the same method also in a case where there are three or more regions with concave down. Also, a boundary can be obtained using the same method also in a case where the inflection point b of FIG. 8 is used as a boundary, in a case where the midpoint c of FIG. 8 is used as a boundary, in a case where the activity state identification unit 16 does not use the region C as a processing target, and in a case where the region C is set as an “intermediate activity state,” which is an activity state different from the region A and the region B. Furthermore, assignment of a specific activity state, such as a seated position, walking, and running, to each region with concave down is not indispensable. Only the number of activity states to be identified may be determined in advance, and such activity states as “activity state A”, “activity state B,” and “activity state C” may be assigned to the regions with concave down, for example.

In addition, although the foregoing has described a case where the position of a maximum of a first derivative of a smoothed histogram is set as a threshold, implementation is possible with use of a roughly similar method in other cases as well, such as a case where the inflection point b, which a minimum of a first derivative, is set as a threshold. Moreover, although the foregoing has described a method of identifying three activity states including a seated position, walking, and running, implementation is possible with use of a roughly similar method also in a case where there are two or four or more activity state.

Also, in a case where smoothing is not sufficient, more than two maxima are created. Although the dash line (C) of FIG. 9 has two maxima as it has been smoothed sufficiently, if more than two points remain as maxima, it is not possible to set one threshold for the seated position and walking, and one threshold for walking and the seated position. Thus, it is necessary to perform processing for setting one threshold for the seated position and walking, and one threshold for walking and running. To this end, with use of an acceleration database of daily lives of a plurality of measurement subjects that has been obtained in advance, an average value of thresholds of the seated position and walking of these measurement subjects (a numerical value 1), as well as an average value of thresholds of walking and running thereof (a numerical value 2), is obtained. Then, a boundary line between the threshold between the seated position and walking and the threshold between walking and the seated position is determined as an average value of the numerical value 1 and the numerical value 2. Furthermore, a lower limit of the threshold between the seated position and walking is set as a numerical value that is always included as the seated position by studying four weeks' worth of data pieces of moving averages of a plurality of labeled measurement subjects that have been chronologically plotted as shown in FIG. 5. Moreover, a case where the number of maxima is smaller than two is handled by, for example, applying an average value of the thresholds of activity states obtained from, for example, labeled data pieces of activity states of a plurality of measurement subjects that have been obtained in advance.

In addition, also in a case where there are two or four or more activity states, that is to say, in a case where there is one or three or more thresholds, a region that is supposed to include a threshold can be set, and a threshold can be set to a maximum that remains until the end in the course of repeating smoothing inside this region.

Also, a case where the number of maxima is smaller than two is handled by, for example, applying an average value of the thresholds of activity states obtained from, for example, labeled data pieces of activity states of a plurality of measurement subject that have been obtained in advance. Note that also in a case where there are two or four or more activity states, an average value of the thresholds of activity states obtained from labeled data pieces and the like can be applied in a similar manner.

The description of FIG. 2 is now resumed. The activity state identification unit 16 identifies activity states in specific periods by comparing the moving average calculated by the moving average calculation unit 13 with the thresholds calculated by the threshold calculation unit 15.

The per-activity state data generation unit 17 categorizes chronological data pieces of biosignals other than the acceleration signal, among the biosignals acquired by the signal acquisition unit 10, by activity state of each period based on the result of identification performed by the activity state identification unit 16.

The per-activity state biosignal storage unit 18 stores the biosignals that the per-activity state data generation unit 17 categorized by activity state of each period.

The stress estimation unit 19 calculates feature values for stress estimation from the biosignals of respective activity states that have been stored in the per-activity state biosignal storage unit 18, and estimates stress with use of the feature values. For example, a model that uses PSS (Perceived Stress Scale) scores as correct answer values of stress and estimates PSS scores through regression analysis can be generated. At this time, scores calculated from a PSS survey of a measurement target that was conducted at the end of an experiment period (four weeks) are used as supervisory data, and feature values disclosed in Non-Patent Document 1, that is to say, for example, the average, variance, median value, and constituent elements of a histogram of the power spectral density of perspiration and body movements, are calculated as stress feature values. Feature values are calculated with respect to data pieces of three respective activity states including the seated position, walking, and running, and also with respect to an entire collection of such data pieces. A machine learning model, such as an SVM model, is trained with use of these feature values. PSS scores can be estimated using the model generated in the aforementioned manner, and can be set as the result of stress estimation.

The stress estimation apparatus 1 outputs the result of stress estimation made by the stress estimation unit 19. Examples of an output method include, but are not limited to, an output via a screen, an output via printing, and the like. Also, depending on a request from the measurement subject, the result of stress estimation may be transmitted to the wearable terminal 50 or a smartphone, and output from a screen pertaining to the wearable terminal 50 or the smartphone.

[Description of Operations]

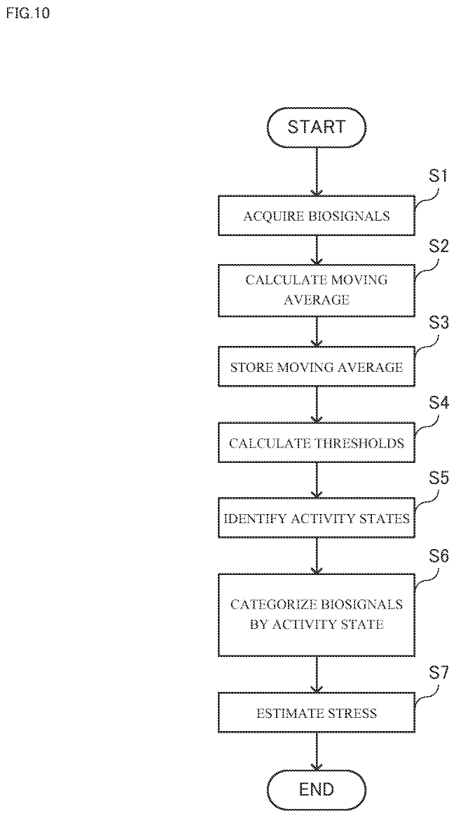

Next, the operations of the stress estimation apparatus 1 in the present example embodiment will be described using FIG. 10. FIG. 10 is a flow diagram showing the operations of the stress estimation apparatus 1. In the following description, FIG. 1 to FIG. 9 will be referred to as appropriate. Also, in the present example embodiment, a stress estimation method is implemented by causing the stress estimation apparatus 1 to operate. Therefore, the following description of the operations of the stress estimation apparatus 1 applies to the stress estimation method in the present example embodiment.

First, the premise is that a measurement subject wears the wearable terminal 50. The wearable terminal detects biological information of the measurement subject, and outputs biosignals. Based on this premise, the signal acquisition unit 10 of the stress estimation apparatus 1 acquires biosignals transmitted from the wearable terminal 50 (S1).

The moving average calculation unit 13 calculates a moving average from an acceleration signal included in the biosignals acquired in S1 (S2). The moving average calculation unit 13 stores the calculated moving average to the moving average storage unit 14 (S3). The threshold calculation unit 15 calculates thresholds from the moving average stored in the moving average storage unit 14 (S4). Specifically, as stated earlier, the threshold calculation unit 15 generates a histogram of the moving average, and obtains two maxima. The threshold calculation unit 15 uses the obtained maxima as a threshold between the seated position and walking and a threshold between walking and the seated position.

The activity state identification unit 16 identifies activity states by comparing biosignals other than the acceleration signal among the biosignals acquired in S1 with the thresholds calculated in S4 (S5). The per-activity state data generation unit 17 categorizes chronological data pieces of the biosignals by activity state (S6). The stress estimation unit 19 calculates stress feature values with use of the categorized chronological data pieces of the biosignals, and estimates stress with use of the stress feature values (S7).

### DESCRIPTION OF ADVANTAGEOUS EFFECTS

In the present example embodiment, activity states can be estimated on an individual basis without using labels of activity states. Thresholds of such activity states as the seated position, walking, and running are estimated on an individual basis by using activity states that have been estimated on an individual basis; this enables more accurate identification of activity states, which is important in stress estimation, and enables stress estimation with higher precision.

[Program]

It is sufficient that a program of the example embodiment of the present invention be a program that causes a computer to execute steps S1 to S7 shown in FIG. 10. The stress estimation apparatus 1 and the stress estimation method in the present example embodiment can be realized by installing this program in the computer and executing this program. In this case, a processor of the computer functions and performs processing as the signal acquisition unit 10, moving average calculation unit 13, threshold calculation unit 15, activity state identification unit 16, per-activity state data generation unit 17, and stress estimation unit 19.

Also, the program in the present example embodiment may be executed by a computer system constructed by a plurality of computers. In this case, for example, each computer may function as one of the signal acquisition unit 10, moving average calculation unit 13, threshold calculation unit 15, activity state identification unit 16, per-activity state data generation unit 17, and stress estimation unit 19.

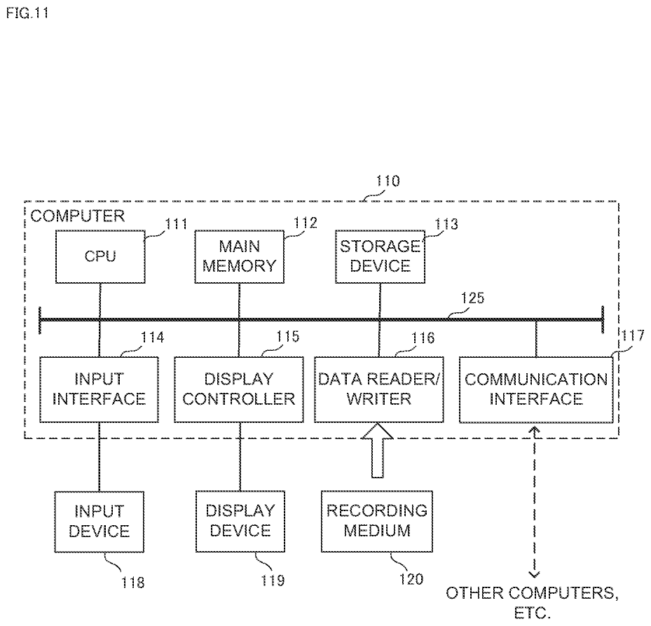

Hereinafter, a computer that realizes the stress estimation apparatus 1 by executing the program in the example embodiment will be described with reference to FIG. 11. FIG. 11 is a block diagram showing one example of a computer that realizes the stress estimation apparatus 1 in the present example embodiment.

As shown in FIG. 11, a computer 110 includes a CPU 111, a main memory 112, a storage device 113, an input interface 114, a display controller 115, a data reader/writer 116, and a communication interface 117. These components are connected in such a manner that they can perform data communication with one another via a bus 125. Note that the computer 110 may include a GPU (Graphics Processing Unit) or an FPGA (Field-Programmable Gate Array) in addition to the CPU 111 or in place of the CPU 111.

The CPU 111 carries out various types of computation by deploying the program (codes) in the present example embodiment stored in the storage device 113 to the main memory 112, and executing the deployed program in a predetermined order. The main memory 112 is typically a volatile storage device, such as a DRAM (Dynamic Random Access Memory). Also, the program in the present example embodiment is provided in a state where it is stored in a computer readable recording medium 120. Note that the program in the present example embodiment may also be distributed over the Internet connected via the communication interface 117.

Furthermore, specific examples of the storage device 113 include a hard disk drive, and also a semiconductor storage device, such as a flash memory. The input interface 114 mediates data transmission between the CPU 111 and an input apparatus 118, such as a keyboard and a mouse. The display controller 115 is connected to a display apparatus 119, and controls displays on the display apparatus 119.

The data reader/writer 116 mediates data transmission between the CPU 111 and the recording medium 120, and executes readout of the program from the recording medium 120, as well as writing of the result of processing in the computer 110 to the recording medium 120. The communication interface 117 mediates data transmission between the CPU 111 and other computers.

Also, specific examples of the recording medium 120 include: a general-purpose semiconductor storage device, such as CF (Compact Flash®) and SD (Secure Digital); a magnetic recording medium, such as Flexible Disk; and an optical recording medium, such as CD-ROM (Compact Disk Read Only Memory).

Note that the stress estimation apparatus 1 in the present example embodiment can also be realized using items of hardware corresponding to respective components, rather than using the computer with the program installed therein. Furthermore, a part of the stress estimation apparatus 1 may be realized by the program, and the remaining part of the stress estimation apparatus 1 may be realized by hardware.

A part or all of the aforementioned example embodiment can be described as, but is not limited to, the following (Supplementary Note 1) to (Supplementary Note 27).

(Supplementary Note 1)

A stress estimation apparatus that estimates stress of a measurement subject, the stress estimation apparatus including:

a signal acquisition unit configured to acquire a biosignal, including acceleration information of an action of at least one part of the measurement subject;

a moving average calculation unit configured to calculate a moving average from the acceleration information included in the biosignal acquired by the signal acquisition unit;

a threshold calculation unit configured to calculate a threshold of an activity state of the measurement subject from the moving average calculated by the moving average calculation unit;

an activity state identification unit configured to identify an activity state in a specific period from the moving average calculated by the moving average calculation unit and the threshold calculated by the threshold calculation unit;

a per-activity state data generation unit configured to generate per-activity state data by categorizing chronological data pieces of the biosignal other than the acceleration information by activity state identified by the activity state identification unit, the biosignal having been acquired by the signal acquisition unit; and

a stress estimation unit configured to estimate stress from the per-activity state data generated by the per-activity state data generation unit.

(Supplementary Note 2)

The stress estimation apparatus according to Supplementary Note 1,

wherein the threshold calculation unit constructs a histogram of the moving average calculated by the moving average calculation unit, and calculates the threshold for the measurement subject with use of the histogram.

(Supplementary Note 3)

The stress estimation apparatus according to Supplementary Note 2,

wherein the threshold calculation unit uses a position of a maximum of a first derivative of a curve obtained by smoothing the histogram as the threshold.

(Supplementary Note 4)

The stress estimation apparatus according to Supplementary Note 2,

wherein the threshold calculation unit uses a position of a minimum of a first derivative of a curve obtained by smoothing the histogram as the threshold.

(Supplementary Note 5)

The stress estimation apparatus according to Supplementary Note 2,

wherein the threshold calculation unit uses a position of a midpoint between neighboring minimum and maximum of a first derivative of a curve obtained by smoothing the histogram as the threshold when the minimum is closer to a zero point.

(Supplementary Note 6)

The stress estimation apparatus according to Supplementary Note 2,

wherein the threshold calculation unit uses both of neighboring minimum and maximum of a first derivative of a curve obtained by smoothing the histogram as the threshold when the minimum is closer to a zero point, and

the activity state identification unit does not use a region between the minimum and the maximum as a processing target.

(Supplementary Note 7)

The stress estimation apparatus according to Supplementary Note 2,

wherein the threshold calculation unit uses both of neighboring minimum and maximum of a first derivative of a curve obtained by smoothing the histogram as the threshold when the minimum is closer to a zero point, and

the activity state identification unit identifies a region between the minimum and the maximum as an activity state region different from that of a neighboring region.

(Supplementary Note 8)

The stress estimation apparatus according to any one of Supplementary Note 3 to Supplementary Note 7,

wherein the threshold calculation unit smooths the constructed histogram after applying logarithmic conversion processing thereto, and

the curve obtained by smoothing the histogram is a curve of the histogram after subjected to the logarithmic conversion processing.

(Supplementary Note 9)

The stress estimation apparatus according to any one of Supplementary Note 3 to Supplementary Note 7,

wherein an extremum of the first derivative of the curve of the histogram is an extremum that remains until the end in the course of increasing a degree of smoothing of a curve of the first derivative in a specific region.

(Supplementary Note 10)

A stress estimation method that estimates stress of a measurement subject, the stress estimation method including:

a step of acquiring a biosignal, including acceleration information of an action of at least one part of the measurement subject;

a step of calculating a moving average from the acceleration information included in the acquired biosignal;

a step of calculating a threshold of an activity state of the measurement subject from the calculated moving average;

a step of identifying an activity state in a specific period from the calculated moving average and the calculated threshold;

a step of generating per-activity state data by categorizing chronological data pieces of the acquired biosignal other than the acceleration information by identified activity state; and

a step of estimating stress from the generated per-activity state data.

(Supplementary Note 11)

The stress estimation method according to Supplementary Note 10,

wherein, in the step of calculating the threshold, a histogram of the calculated moving average is constructed, and the threshold for the measurement subject is calculated with use of the histogram.

(Supplementary Note 12)

The stress estimation method according to Supplementary Note 11,

wherein, in the step of calculating the threshold, a position of a maximum of a first derivative of a curve obtained by smoothing the histogram is used as the threshold.

(Supplementary Note 13)

The stress estimation method according to Supplementary Note 11,

wherein, in the step of calculating the threshold, a position of a minimum of a first derivative of a curve obtained by smoothing the histogram is used as the threshold.

(Supplementary Note 14)

The stress estimation method according to Supplementary Note 11,

wherein, in the step of calculating the threshold, a position of a midpoint between neighboring minimum and maximum of a first derivative of a curve obtained by smoothing the histogram is used as the threshold when the minimum is closer to a zero point.

(Supplementary Note 15)

The stress estimation method according to Supplementary Note 11,

wherein, in the step of calculating the threshold, both of neighboring minimum and maximum of a first derivative of a curve obtained by smoothing the histogram are used as the threshold when the minimum is closer to a zero point, and

in the step of identifying the activity state, a region between the minimum and the maximum is not used as a processing target.

(Supplementary Note 16)

The stress estimation method according to Supplementary Note 11,

wherein, in the step of calculating the threshold, both of neighboring minimum and maximum of a first derivative of a curve obtained by smoothing the histogram are used as the threshold when the minimum is closer to a zero point, and

in the step of identifying the activity state, a region between the minimum and the maximum is identified as an activity state region different from that of a neighboring region.

(Supplementary Note 17)

The stress estimation method according to any one of Supplementary Note 12 to Supplementary Note 16,

wherein, in the step of calculating the threshold, the constructed histogram is smoothed after applying logarithmic conversion processing thereto, and

the curve obtained by smoothing the histogram is a curve of the histogram after subjected to the logarithmic conversion processing.

(Supplementary Note 18)

The stress estimation method according to any one of Supplementary Note 12 to Supplementary Note 16,

wherein, an extremum of the first derivative of the curve of the histogram is an extremum that remains until the end in the course of increasing a degree of smoothing of a curve of the first derivative in a specific region.

(Supplementary Note 19)

A computer readable recording medium that includes a program recorded thereon, the program causing a computer to estimate stress of a measurement subject and causing the computer to carry out:

a step of acquiring a biosignal, including acceleration information of an action of at least one part of the measurement subject;

a step of calculating a moving average from the acceleration information included in the acquired biosignal;

a step of calculating a threshold of an activity state of the measurement subject from the calculated moving average;

a step of identifying an activity state in a specific period from the calculated moving average and the calculated threshold;

a step of generating per-activity state data by categorizing chronological data pieces of the acquired biosignal other than the acceleration information by identified activity state; and

a step of estimating stress from the generated per-activity state data.

(Supplementary Note 20)

The computer readable recording medium according to Supplementary Note 19,

wherein, in the step of calculating the threshold, a histogram of the calculated moving average is constructed, and the threshold for the measurement subject is calculated with use of the histogram.

(Supplementary Note 21)

The computer readable recording medium according to Supplementary Note 20,

wherein, in the step of calculating the threshold, a position of a maximum of a first derivative of a curve obtained by smoothing the histogram is used as the threshold.

(Supplementary Note 22)

The computer readable recording medium according to Supplementary Note 20,

wherein in the step of calculating the threshold, a position of a minimum of a first derivative of a curve obtained by smoothing the histogram is used as the threshold.

(Supplementary Note 23)

The computer readable recording medium according to Supplementary Note 20,

wherein, in the step of calculating the threshold, a position of a midpoint between neighboring minimum and maximum of a first derivative of a curve obtained by smoothing the histogram is used as the threshold when the minimum is closer to a zero point.

(Supplementary Note 24)

The computer readable recording medium according to Supplementary Note 20,

wherein, in the step of calculating the threshold, both of neighboring minimum and maximum of a first derivative of a curve obtained by smoothing the histogram are used as the threshold when the minimum is closer to a zero point, and

in the step of identifying the activity state, a region between the minimum and the maximum is not used as a processing target.

(Supplementary Note 25)

The computer readable recording medium according to Supplementary Note 20,

wherein, in the step of calculating the threshold, both of neighboring minimum and maximum of a first derivative of a curve obtained by smoothing the histogram are used as the threshold when the minimum is closer to a zero point, and

in the step of identifying the activity state, a region between the minimum and the maximum is identified as an activity state region different from that of a neighboring region.

(Supplementary Note 26)

The computer readable recording medium according to any one of Supplementary Note 21 to Supplementary Note 25,

wherein, in the step of calculating the threshold, the constructed histogram is smoothed after applying logarithmic conversion processing thereto, and

the curve obtained by smoothing the histogram is a curve of the histogram after subjected to the logarithmic conversion processing.

(Supplementary Note 27)

The computer readable recording medium according to any one of Supplementary Note 21 to Supplementary Note 25,

wherein, an extremum of the first derivative of the curve of the histogram is an extremum that remains until the end in the course of increasing a degree of smoothing of a curve of the first derivative in a specific region.

While the present invention has been described above with reference to the example embodiment, the present invention is not limited to the aforementioned example embodiment. Various changes that can be understood by a person skilled in the art within the scope of the present invention can be made to the configurations and details of the present invention.

This application is based upon and claims the benefit of priority from Japanese application No. 2019-73714, filed on Apr. 8, 2019, the disclosure of which is incorporated herein in its entirety by reference.

### REFERENCE SIGNS LIST

1 stress estimation apparatus
10 signal acquisition unit
11 acceleration signal obtainment unit
12 unit that acquires biosignals other than acceleration
13 moving average calculation unit
14 moving average storage unit
15 threshold calculation unit
16 activity state identification unit
17 per-activity state data generation unit
18 per-activity state biosignal storage unit
19 stress estimation unit
50 wearable terminal
110 computer
111 CPU
112 main memory
113 storage device
114 input interface
115 display controller
116 data reader/writer
117 communication interface
118 input apparatus
119 display apparatus
120 recording medium
125 bus

What is claimed is:

1. A stress estimation apparatus that estimates stress of a measurement subject, the stress estimation apparatus comprising:
a signal acquisition unit configured to acquire a biosignal, including acceleration information of an action of at least one part of the measurement subject;
a moving average calculation unit configured to calculate a moving average from the acceleration information included in the biosignal acquired by the signal acquisition unit;
a threshold calculation unit configured to calculate a threshold of an activity state of the measurement subject from the moving average calculated by the moving average calculation unit;
an activity state identification unit configured to identify an activity state in a specific period from the moving average calculated by the moving average calculation unit and the threshold calculated by the threshold calculation unit;
a per-activity state data generation unit configured to generate per-activity state data by categorizing chronological data pieces of the biosignal other than the acceleration information, out of the biosignal acquired by the signal acquisition unit, by activity state identified by the activity state identification unit; and
a stress estimation unit configured to estimate stress from the per-activity state data generated by the per-activity state data generation unit.

2. The stress estimation apparatus according to claim 1,
wherein the threshold calculation unit constructs a histogram of the moving average calculated by the moving average calculation unit, and calculates the threshold for the measurement subject with use of the histogram.

3. The stress estimation apparatus according to claim 2,
wherein the threshold calculation unit uses a position of a maximum of a first derivative of a curve obtained by smoothing the histogram as the threshold.

4. The stress estimation apparatus according to claim 2,
wherein the threshold calculation unit uses a position of a minimum of a first derivative of a curve obtained by smoothing the histogram as the threshold.

5. The stress estimation apparatus according to claim 2,
wherein the threshold calculation unit uses a position of a midpoint between neighboring minimum and maximum of a first derivative of a curve obtained by smoothing the histogram as the threshold when the minimum is closer to a zero point.

6. The stress estimation apparatus according to claim 2,
wherein the threshold calculation unit uses both of neighboring minimum and maximum of a first derivative of a curve obtained by smoothing the histogram as the threshold when the minimum is closer to a zero point, and
the activity state identification unit does not use a region between the minimum and the maximum as a processing target.

7. The stress estimation apparatus according to claim 2,
wherein the threshold calculation unit uses both of neighboring minimum and maximum of a first derivative of a curve obtained by smoothing the histogram as the threshold when the minimum is closer to a zero point, and
the activity state identification unit uses identifies a region between the minimum and the maximum as an activity state region different from that of a neighboring region.

8. The stress estimation apparatus according to claim 3,
wherein the threshold calculation unit smooths the constructed histogram after applying logarithmic conversion processing thereto, and
the curve obtained by smoothing the histogram is a curve of the histogram after subjected to the logarithmic conversion processing.

9. A stress estimation method that estimates stress of a measurement subject, the stress estimation method comprising:
acquiring a biosignal, including acceleration information of an action of at least one part of the measurement subject;
calculating a moving average from the acceleration information included in the acquired biosignal;
calculating a threshold of an activity state of the measurement subject from the moving average;
identifying an activity state in a specific period from the moving average and the calculated threshold;
generating per-activity state data by categorizing chronological data pieces of the biosignal other than the acceleration information, out of the acquired biosignal, by the identified activity state; and
estimating stress from the generated per-activity state data.

10. A non-transitory computer readable recording medium that includes a program recorded thereon, the program including commands for causing a computer to estimate stress of a measurement subject and causing the computer to carry out:
acquiring a biosignal, including acceleration information of an action of at least one part of the measurement subject;
calculating a moving average from the acceleration information included in the acquired biosignal;
calculating a threshold of an activity state of the measurement subject from the calculated moving average;
identifying an activity state in a specific period from the calculated moving average and the calculated threshold;
generating per-activity state data by categorizing chronological data pieces of the biosignal other than the acceleration information, out of the acquired biosignal, by the identified activity state; and
estimating stress from the generated per-activity state data.

11. The stress estimation method according to claim 9,
wherein, in the calculating the threshold, a histogram of the calculated moving average is constructed, and the threshold for the measurement subject is calculated with use of the histogram.

12. The stress estimation method according to claim 11,
wherein, in the calculating the threshold, a position of a maximum of a first derivative of a curve obtained by smoothing the histogram is used as the threshold.

13. The stress estimation method according to claim 11,
wherein, in the calculating the threshold, a position of a minimum of a first derivative of a curve obtained by smoothing the histogram is used as the threshold.

14. The stress estimation method according to claim 11,
wherein, in the calculating the threshold, a position of a midpoint between neighboring minimum and maximum of a first derivative of a curve obtained by smoothing the histogram is used as the threshold when the minimum is closer to a zero point.

15. The stress estimation method according to claim 11,
wherein, in the calculating the threshold, both of neighboring minimum and maximum of a first derivative of a curve obtained by smoothing the histogram are used as the threshold when the minimum is closer to a zero point, and
in the identifying the activity state, a region between the minimum and the maximum is not used as a processing target.

16. The non-transitory computer readable recording medium according to claim 10,
wherein, in the calculating the threshold, a histogram of the calculated moving average is constructed, and the threshold for the measurement subject is calculated with use of the histogram.

17. The non-transitory computer readable recording medium according to claim 16,
wherein, in the calculating the threshold, a position of a maximum of a first derivative of a curve obtained by smoothing the histogram is used as the threshold.

18. The non-transitory computer readable recording medium according to claim 16,
wherein, in the calculating the threshold, a position of a minimum of a first derivative of a curve obtained by smoothing the histogram is used as the threshold.

19. The non-transitory computer readable recording medium according to claim 16,
wherein, in the calculating the threshold, a position of a midpoint between neighboring minimum and maximum of a first derivative of a curve obtained by smoothing the histogram is used as the threshold when the minimum is closer to a zero point.

20. The non-transitory computer readable recording medium according to claim 16,
wherein, in the calculating the threshold, both of neighboring minimum and maximum of a first derivative of a curve obtained by smoothing the histogram are used as the threshold when the minimum is closer to a zero point, and
in the identifying the activity state, a region between the minimum and the maximum is not used as a processing target.
